(19) United States
(12) Patent Application Publication
(10) Pub. No.: US 2023/0165608 A1
(43) Pub. Date: Jun. 1, 2023

(54) HEIGHT RESTORING DEVICE, INSERTION APPARATUS HAVING A HEIGHT RESTORING DEVICE AND METHOD FOR RESTORING THE HEIGHT OF AND STABILISING THE SPINAL COLUMN

(71) Applicant: Joline GmbH & Co. KG, Hechingen (DE)

(72) Inventors: Peter Kohlbecher, Engen (DE); Jan Maier, Mössingen (DE)

(73) Assignee: Joline GmbH & Co. KG, Hechingen (DE)

(21) Appl. No.: 17/796,371

(22) PCT Filed: Jan. 29, 2021
(86) PCT No.: PCT/EP2021/052205

§ 371 Date: Jan. 25, 2023

(30) Foreign Application Priority Data
Jan. 29, 2020 (DE) 10 2020 102 100.3
May 15, 2020 (DE) 10 2020 113 300.6

(51) Int. Cl. A61B 17/70; A61B 17/88

(52) U.S. Cl. CPC A61B 17/7098; A61B 17/8816

(57) ABSTRACT

The invention relates to a height restoring device for restoring the height of and stabilizing the spinal column, in particular for stabilizing broken vertebral bodies or contused intervertebral discs, which device can be arranged in an insertion position in a compression-fractured vertebral body or between adjacent vertebral bodies and once there, can be transferred from the insertion position into an expansion position by means of an expansion apparatus.

### BACKGROUND

The invention relates to a height restoring device for restoring the height of and stabilizing the spinal column, in particular for stabilizing broken vertebral bodies or contused intervertebral discs, which device can be arranged in an insertion position in a compression-fractured vertebral body or between adjacent vertebral bodies and once there, can be transferred from the insertion position into an expansion position by means of an expansion apparatus.

Height restoring devices are known from the prior art, which are used to restore the height of and stabilize broken vertebral bodies. Devices of this kind may comprise a catheter that can be inserted into the vertebral cavity through a passage drilled in the pedicle of the broken vertebra. It is also known to guide a pressure balloon, which can be expanded via a pressure line, through the catheter into the cavity of the vertebra, as a result of which a vertebral body that was contused or possibly broken by compression can be expanded again and returned to the original shape thereof. It is also known to remove the pressure balloon after the height of the vertebral body was restored and to fill the cavity created by the pressure balloon with hardening bone filling material.

It is known from EP 1 308 134 B1 to provide a support body that is made of expanded metal and has an expandable wall in order to prevent the cavity created by the body from being compressed again after the pressure balloon has been removed. However, it has been shown that the expanded metal does not always reliably retain the expansion position thereof after the pressure balloon has been removed, since the forces acting on the support body may be comparatively high.

US Patent US 2016/0302942 A1 describes a height restoring device having features of the preamble of claim 1. The support body shown there has a central portion and an upper and lower bone contact structure, with the central portion being shifted axially in order to shift the support body into the expansion position, as a result of which the bone contact structures are shifted upward and downward. The length extension of the support body remains unchanged during the height restoring process.

Other similar devices are known from U.S. Pat. No. 6,127,597, US 2012/0071977, US 2009/0204216 A1, and WO-A-0176514.

EP 2405835B1 discloses an implant that has a support body having position surfaces that are held in an expansion position by means of pairs of supports. Furthermore, a proximal and a distal end portion are provided, the pairs of supports being assigned to the end portions and the end portions being moved toward one another when shifted into the expansion position. For this purpose, the individual supports of the pairs of supports each have a first engagement element and a second engagement element, the engagement elements of the supports of the pairs of supports engaging with one another.

### SUMMARY OF THE INVENTION

The object of the present invention is to provide a height restoring device and an insertion apparatus having a height restoring device with which a safe expansion of the support body is ensured. In particular, the aim is to ensure that the cavity created by the height restoring device in the expansion position is retained during and after the expansion and in particular before any filling material is introduced into the cavity, and that the height restoring device is as slim as possible in the insertion position and as short as possible in the expansion position. In addition, it is desirable to expand the height restoring device from the insertion position into the expansion position by means of a pressure balloon that can be arranged inside the height restoring device.

This object is achieved by a height restoring device having the features of claim 1.

Consequently, it is provided in particular that, when the support body is shifted into the expansion position, the end portions move toward one another along the longitudinal axis and the support portions move radially outward relative to the carrier portions, so that their latching portions act against the counter-latching portions of the carrier portions for latching.

Thus, when expanding the support body, both the support portions and the carrier portions are shifted radially outward relative to one another, with the relative movement resulting in a secure latching of the latching portions into the counter-latching portions. Furthermore, a cavity is created in the central region of the support body, i.e., in the region of the central longitudinal axis. On the one hand, a pressure balloon can unfold in the cavity to expand the support body and, on the other hand, the cavity can be filled with bone cement after the pressure balloon has been removed.

The carrier portions and/or the support portions are preferably designed at least in portions as resiliently yielding webs or bands or in a web-like or band-like manner, in particular at least in the portions or regions in which they act against one another.

Preferably, four, five, six, or more carrier portions and a corresponding number of support portions are provided, with the individual carrier portions and support portions each being symmetrical to the longitudinal axis. This has the advantage that it is not necessary to pay attention to top, bottom, right, or left when inserting the height restoring device.

Due to the latching fixation, it can be achieved that the support portions act directly against the inner sides of the carrier portions and support the carrier portions in a stabilizing manner not only in the expansion position but also during the expansion of the support body, so that they can stably retain their expansion position after the expansion has ended or after the expansion apparatus has been switched off and in particular, when using pressure balloons, after removing the pressure balloons.

The latching fixation forms in this case in particular a kind of free-running locking mechanism which causes the carrier portions to move into the expansion position, i.e., in the free-running direction, but blocks the return movement of the carrier portions into the insertion position due to the latching.

It is provided that the support portions have latching portions and that the carrier portions have counter-latching portions, so that the latching portions act against the counter-latching portions for latching fixation of the carrier portions. Overall, an at least largely form-fitting interaction of latching portions and counter-latching portions can be achieved in this way, the arrangement preferably being such that, during further expansion, the support portions or their latching portions interact with the counter-latching portions in such a way that reliable support is achieved by the latching, and a movement of the carrier portions back in the direction of the insertion position is blocked during the entire widening process of the support body.

The individual latching portions and/or counter-latching portions can be designed as several latching elements lying one behind the other and/or next to one another. For this, in particular latching webs, openings, recesses, impressions, corrugations, projections, pawls, teeth, and/or saw teeth come into consideration as latching elements. In this way, in particular, a free-running locking mechanism can be implemented in an advantageous manner.

Even if latching portions and counter-latching portions are discussed here, this should also mean friction portions and counter-friction portions. The friction portions and/or counter-friction portions can in particular have a correspondingly rough surface or a friction lining with a suitable grain size or coating.

Furthermore, it is advantageous that the carrier portions are arranged radially on the outside and the support portions are arranged radially on the inside. The carrier portions can consequently cover the support portions, in particular in the insertion position. It is advantageous if the latching portions are provided on the radially outer side of the support portions and/or if the counter-latching portions are provided on the radially inner side of the carrier portions.

The support portions and also the carrier portions are preferably designed to be resiliently yielding such that they move radially outward at least in portions when shifted into the expansion position, and that the mutually facing sides of the support portions and carrier portions can interact with one another for latching fixation.

Furthermore, it has proven to be advantageous if the support portions are provided on the respective end portions or are formed onto them or are connected to them. As a result, they can assume a defined position, so that, in the expansion position, they can reliably dissipate forces acting on the carrier portions via the end portions into the support body.

It has also turned out to be advantageous if a structure is provided between the carrier portions and the end portions, which structure shortens the distance between the carrier portions and the end portions when shifted into the expansion position. The shortening can be brought about or increased by the relative movement between the carrier portions and the support portions. The relative movement results in the latching of the respective latching portions into the respective counter-latching portions. The structure can be formed from an expanded or support mesh, for example.

It has been found to be preferable if the structure is formed by connecting webs extending in the axial direction between the individual carrier portions and the corresponding end portion, in particular in the insertion position. Two connecting webs each which, in the insertion position, extend in the axial direction may be provided between each carrier portion, each designed in particular also in a web-like manner, and the corresponding end portion. In the insertion position, the connecting webs then lie next to one another in a circular path about a longitudinal axis. The connecting webs serve in particular to guide the carrier portions when shifted into the expansion position. Due to the comparatively filigree design of the connecting webs, the connecting webs are not used in particular to support the carrier portions; the support portions which act against the inner side of the carrier portions are provided for this purpose.

In order to achieve a suitable shortening of the distance between the carrier portions and the end portions when shifted into the expansion position, it is conceivable that the connecting webs of respectively adjacent carrier portions are connected to one another, in particular at connection points, or that adjacent carrier portions at least partially provide common connecting webs. The connecting webs can be spread apart when shifted into the expansion position, as a result of which the distance between the carrier portions and the end portions is shortened overall and thus a relative movement between the carrier portions and the support portions is caused. The relative movement ultimately results in the latching of the respective latching portions into the respective counter-latching portions.

It is also conceivable that the support portions each have at least two partial support portions having latching portions, and that an intermediate portion is provided between the partial support portions, which intermediate portion is not expanded radially outward or is expanded less than the partial support portions when it is transferred into the expansion position. An arrangement of this kind is comparatively space-saving, in particular in the insertion position, and can nevertheless result in reliable support of the carrier portions. An intermediate portion is advantageously provided centrally between the two end portions. However, it is also conceivable that two or more intermediate portions are provided. The provision of such an intermediate portion contributes to a relative movement taking place between the carrier portions and the support portions during expansion.

The at least one intermediate portion may be formed by a sleeve or a ring, both of which do not noticeably deform when shifted into the expansion position. The design with an intermediate portion as a sleeve or ring also has the advantage that, in the expansion position, forces acting on the carrier portions perpendicular to the central axis can be advantageously dissipated via the support portions that then enclose comparatively small angles with a plane running perpendicular to the central axis. It is also conceivable that the intermediate portion is formed by expanded or support mesh. The intermediate portion is preferably formed from connecting webs, at least two connecting webs which, in the insertion position, extend in the axial direction being provided on the respective partial support portions, and the connecting webs of respectively adjacent partial support portions being connected to one another in particular via connection points, or adjacent partial support portions at least partially providing common connecting webs. In particular in the insertion position, the connecting webs then lie next to one another in a circular path about a longitudinal axis. The connecting webs can be spread apart when shifted into the expansion position, as a result of which the distance between the partial support portions is shortened overall and thus an additional relative movement between the carrier portions and the support portions is caused. The relative movement ultimately results in the latching of the respective latching portions into the respective counter-latching portions.

A further embodiment provides that the carrier portions are connected to the support portions at the proximal end portion and at the distal end portion. Overall, this results in a simple structure.

It is also advantageous if the support body has a two-layer structure concentric about a longitudinal axis in the insertion position, the inner part being formed by the support portions and the end portions, and the outer part being formed by the carrier portions and in particular also by the structure. In addition, it is advantageous if, in the insertion position, the support portions are arranged running substantially parallel to the carrier portions in the axial direction, and it is advantageous that the carrier portions at least largely enclose the support portions. A structure of this kind can be built comparatively slim and is therefore suitable due to the fact that the height restoring device, in the insertion position, can be shifted in a space-saving manner to the intended expansion site in the body by means of an insertion catheter.

With the expansion apparatus, fillable pressure balloons can be placed in particular between the end portions, the intermediate portion, and the corresponding end portion and/or between the adjacent intermediate portions such that the corresponding pressure balloon dilates for transfer to the expansion position and that, when the corresponding pressure balloon dilates, the carrier portions are moved in the radial direction, and at the same time the support portions act in a latching manner against the carrier portions to fix the carrier portions. The provision of a plurality of pressure balloons, which may be arranged one behind the other in particular along the axial direction, has the advantage that these can preferably be dilated independently of one another, so that a functionally reliable and reproducible expansion and thus widening of the space in the corresponding vertebral body or between the vertebral bodies can be achieved.

Advantageously, the pressure balloons can be removed after the expansion position has been reached, and the cavity of the expanded support body can be filled with hardening bone filling material. Due to the latching fixation of the support portions, the pressure balloons can consequently be removed without the support body and the cavity created by the support body being pressed together or compressed again. Forces can be safely absorbed via the expanded support body, and the cavity created by the support body is retained, so that in particular hardening filling material can be introduced into the cavity. The height restoring device is in this case preferably designed as an implant and remains in the body.

According to the invention, it is also conceivable that the proximal and/or the distal end portion has/have a through hole having an inner thread for screwing in a fixing screw after the expansion position has been reached. The expansion position can consequently be secured with the fixing screw. The securing can take place until the cavity created by the support body is filled with filling material. However, it is also conceivable for the expansion position to be permanently secured with the fixing screw, in which case the cavity does not have to be filled with filling material.

Furthermore, it is advantageous if a net or honeycomb structure is provided between the end portions and the corresponding intermediate portion and/or between the adjacent intermediate portions. In particular, a fillable pressure balloon can then be placed within the corresponding net or honeycomb structure. In particular, the net or honeycomb structure results in a homogeneous and stable overall surface, as a result of which the height restoring device can be designed more stable overall. In addition, the implant can have a positive effect on the surroundings and not partially result in traumatization.

The height restoring device can be made of titanium, stainless steel, CoCr, or similar implant materials. It is also conceivable that different materials are used. A combination of different materials with different manufacturing processes is also possible. In particular, it is conceivable for the device, or parts thereof, to be manufactured by means of additive manufacturing methods, as a result of which the carrier portions and the support portions in particular can be partially reinforced.

The object mentioned at the outset is also achieved by an insertion apparatus having an insertion cannula and a height restoring device according to the invention provided in or at the free end of the insertion cannula, which height restoring device can be inserted into a compression-fractured vertebral body or between adjacent vertebral bodies, the insertion cannula actuating the expansion apparatus, and, if necessary, the support body being filled with filling material or the fixing screw being screwed in. The expansion apparatus and preferably one or more fillable pressure balloons can consequently be actuated via the insertion cannula, as a result of which the height restoring device or the support body can be shifted from the insertion position into the expansion position. After the expansion position has been reached, the insertion apparatus or the pressure balloons can be removed from the height restoring device via the insertion cannula, and the cavity created by the support body can be filled with filling material, or the fixing screw can be introduced. The insertion apparatus is designed in such a way that the diameter of the insertion cannula corresponds at least largely to the diameter of the height restoring device in the insertion position. Such an insertion apparatus can, for example, be brought to the expansion site by means of an insertion catheter, in which site the height of the vertebral body or bodies in question is to be restored.

It is also advantageous if a shiftable inner cannula is provided on the inner wall of the insertion cannula, which inner cannula is coupled to the height restoring device in such a way that the height restoring device is detached from the insertion cannula by moving the inner cannula in the distal direction. The height restoring device can thus be decoupled from the outer cannula via the inner cannula. The decoupling is particularly advantageous before the pressure balloons are dilated, because the height restoring device expands in the radial direction and at the same time the length thereof in the axial direction is reduced when the pressure balloons are dilated. Due to the decoupling of the height restoring device and the outer cannula, the height restoring device can expand freely.

Advantageously, the insertion apparatus comprises a stylet having a cutting tip, which stylet can be passed through the insertion cannula and through the height restoring device, and the cutting tip of which forms the distal end of the insertion apparatus in an insertion configuration, so that the height restoring device can be inserted directly into a vertebral body for restoring the height of the corresponding vertebral body, or between adjacent vertebral bodies, i.e., for restoring the height of an existing intervertebral disc between the respective vertebral bodies. This embodiment has the advantage that the insertion apparatus, and in particular the height restoring device, does not have to be brought to the intended location by an insertion catheter, but rather can be moved directly through the corresponding tissue to the intended expansion site without having to be moved via such an insertion catheter. An insertion catheter is consequently not necessary.

To actuate the height restoring device, it is advantageous if an actuating apparatus is provided at the proximal end of the insertion apparatus, with which actuating apparatus the inner cannula can be shifted in the distal direction to detach the height restoring device, and/or with which the inner cannula can be inserted into the insertion cannula or removed from it, and/or with which the stylet can be inserted into or removed from the inner cannula, and/or with which the height restoring device can be brought to the corresponding expansion site in the body.

The object mentioned at the outset is also achieved by a method for restoring the height of and stabilizing the spinal column, in particular for stabilizing broken vertebral bodies or contused intervertebral discs using an insertion catheter according to the invention, the height restoring device being introduced in the insertion position into a vertebral body or between vertebral bodies into the expansion site, and there the height restoring device being transferred from the insertion position into the expansion position. Such a method can be used to restore the height of and stabilize the spinal column in a comparatively simple and yet functionally reliable manner.

In particular, the method can provide that, before the expansion of the height restoring device, an access to the expansion site in the body is created, through which access the height restoring device extends to the expansion site. Access can be created in this case as follows:
introducing an access instrument, in particular via a pedicle, into the expansion site,
placing a pin guide at the expansion site, in particular through the access instrument, and removing the access instrument, the pin guide extending from the expansion site to the outside of the body,
inserting a drill cannula via the pin guide, the drill cannula comprising a cannula and a drill that can be detached from the cannula, and drilling open the vertebral body at the expansion site,
removing the drill, so that the cannula remains in the body and access to the expansion site is established through the cannula.

In this case, the cannula has an inner diameter that is dimensioned so that the height restoring device can be inserted through the access formed by the cannula into the expansion site.

Creating the access first and then inserting the height restoring device through the access is advantageous in particular when the insertion apparatus does not comprise a stylet having a cutting tip, with which stylet the height restoring device can be introduced directly into a vertebral body or between adjacent vertebral bodies.

However, if the insertion apparatus has a stylet having a cutting tip, the height restoring device can also be inserted directly into a vertebral body or between adjacent vertebral bodies into the expansion site.

The method can further provide for at least one pressure balloon to be inserted through the cannula into the expansion site and to be dilated to widen the expansion site and to create a cavity before the height restoring device is inserted into the access.

In particular, the method further provides that, after the expansion site has been widened and the at least one pressure balloon has been removed, the height restoring device is inserted through the access into the expansion site.

In particular, the method also provides that after the insertion of the height restoring device into the expansion site pressure balloons placed in the height restoring device are dilated for transferring the height restoring device into the expansion position, and that the pressure balloons are removed after the height restoring device has reached the expansion position.

It may also be provided for bone filling material to be introduced into the support body after the pressure balloons have been removed.

However, it is also conceivable that the height restoring device is pulled back in the proximal direction before the pressure balloons are dilated in the height restoring device, and the pressure balloons are introduced into the cavity created in the distal direction behind the height restoring device and dilated to create a cavity, and then either the cavity is filled with filling material immediately after the pressure balloons have been removed or the height restoring device is introduced into the cavity and expanded there. If it turns out that the cavity as such is stable enough, the procedure can be in such a way that the cavity is filled directly with bone filling material or bone cement without the height restoring device being introduced into the cavity and expanded there. A procedure of this kind has the advantage that it is possible to align and restore the height of the spinal column without the height restoring device remaining in the body. However, should the cavity prove not to be sufficiently stable, the height restoring device can be inserted into the cavity by being pushed distally and expanded there before bone filling material is introduced through the insertion cannula into the vertebra or between the vertebral bodies.

In this case, it can additionally be provided for the height restoring device to be detached from the insertion cannula before the pressure balloons are dilated, in particular in order to allow the height restoring device to expand unhindered.

Furthermore, it is advantageous if the insertion apparatus is introduced into a vertebral body or between vertebral bodies in the insertion configuration, i.e., with the stylet provided in the inner cannula, which stylet has the cutting tip at the distal end thereof. Accordingly, the insertion through an insertion catheter can be omitted since the insertion apparatus is introduced directly into the body using the stylet.

### BRIEF DESCRIPTION OF THE DRAWINGS

Further details and advantageous configurations of the invention can be found in the following description, on the basis of which embodiments of the invention are described and explained in more detail.

In the drawings:

FIG. 1 shows a first height restoring device in the expansion position;

FIG. 1a is an enlarged longitudinal section of detail Ia from FIG. 1;

FIG. 1b shows the inner part of the height restoring device according to FIG. 1;

FIG. 1c shows the outer part of the height restoring device according to FIG. 1;

FIG. 2 shows the height restoring device according to FIG. 1 in the insertion position;

FIG. 3a is a side view of the height restoring device according to FIG. 1 in the insertion position;

FIG. 3b shows a longitudinal section through the height restoring device according to FIG. 3a;

FIG. 4a is a side view of another height restoring device in the insertion position;

FIG. 4b is a longitudinal section through the height restoring device according to FIG. 4a;

FIG. 5a shows a further embodiment of a height restoring device in the expansion position;

FIG. 5b shows the inner part of the height restoring device according to FIG. 5a;

FIG. 6 is a side view of a further embodiment of a height restoring device in the insertion position;

FIG. 7 shows a longitudinal section through the height restoring device according to FIG. 6;

FIG. 8 shows the device according to FIGS. 6 and 7 in the expansion position;

FIG. 9 shows the detail IX from FIG. 8 in an enlarged representation in longitudinal section;

FIG. 10 is a perspective view of an insertion apparatus;

FIG. 11 is the top view of the insertion apparatus according to FIG. 10;

FIG. 12 is the longitudinal section through the insertion apparatus according to FIG. 11 along the line XII;

FIG. 13 shows an enlargement of section XIII from FIG. 12;

FIG. 14 shows the insertion apparatus according to FIGS. 10 to 12 with the stylet pulled back;

FIG. 15 shows the insertion apparatus according to FIGS. 10 to 12 without stylet;

FIG. 16 shows the insertion apparatus according to FIG. 15 with the height restoring device detached;

FIG. 17 shows the insertion apparatus according to FIG. 16 without an inner cannula;

FIG. 18 is the side view of another insertion apparatus;

FIG. 19 is the top view of the insertion apparatus according to FIG. 18

FIG. 20 shows a longitudinal section through the insertion apparatus according to FIG. 18 along the line XX;

FIG. 21 shows an enlargement of section XXI from FIG. 20;

FIG. 22 shows an enlargement of section XXII from FIG. 20;

FIG. 23 is a perspective view of the actuating apparatus from FIG. 22;

FIG. 24 is a perspective view of the partially dismantled actuating apparatus from FIG. 23, and

FIG. 25 is a schematic representation of the introduction of the height restoring device into the expansion site.

### DETAILED DESCRIPTION

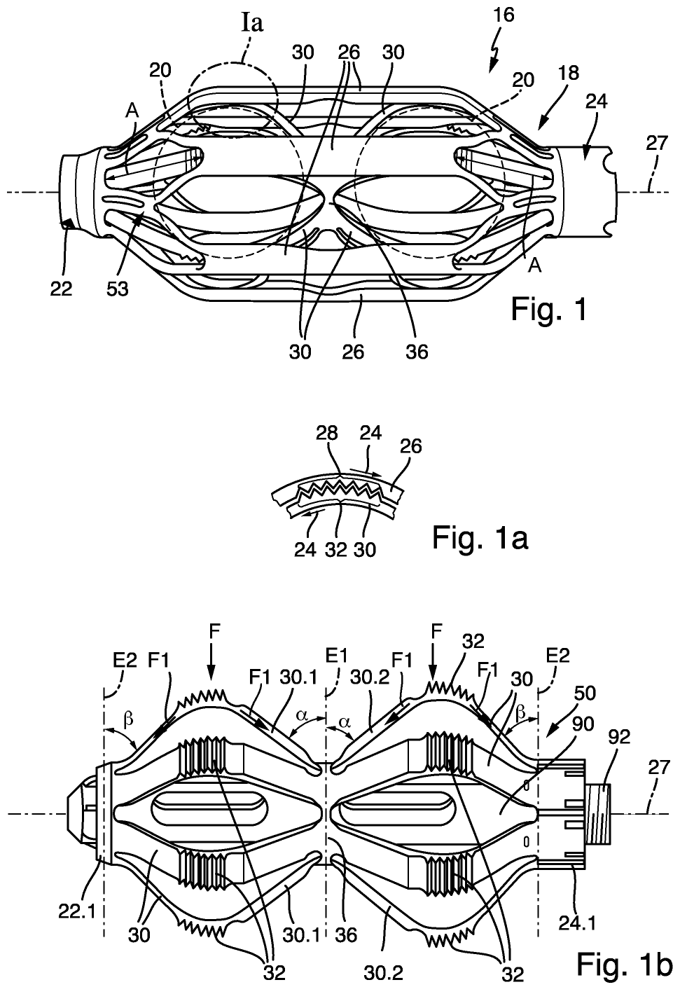

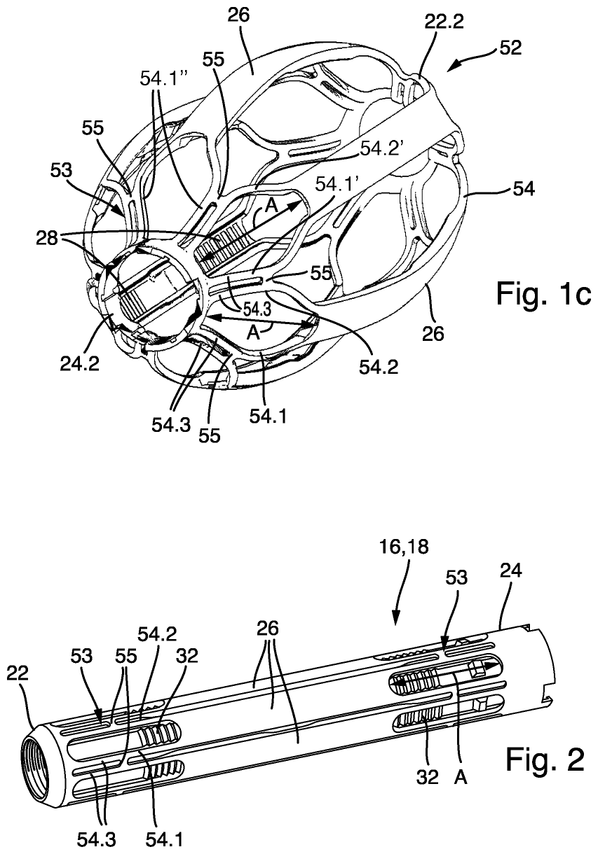

FIG. 1 shows a height restoring device 16 for restoring the height of and stabilizing the spinal column, in particular for stabilizing and restoring the height of broken vertebral bodies or contused intervertebral discs.

The height restoring device 16 has a support body 18 which, in an insertion position as shown in FIG. 2, can be inserted directly or through a catheter into a compression-fractured vertebral body or into the region of an intervertebral disc between adjacent vertebral bodies. To restore the height of the broken vertebral bodies or a contused spinal column or contused intervertebral discs, the height restoring device 16 can be transferred from the insertion position shown in FIG. 2 into the expansion position shown in FIG. 1.

In order to transfer the height restoring device 16 into the expansion position, pressure balloons 20 which can be filled and which are indicated in FIG. 1 by dashed lines may be provided within the support body 18.

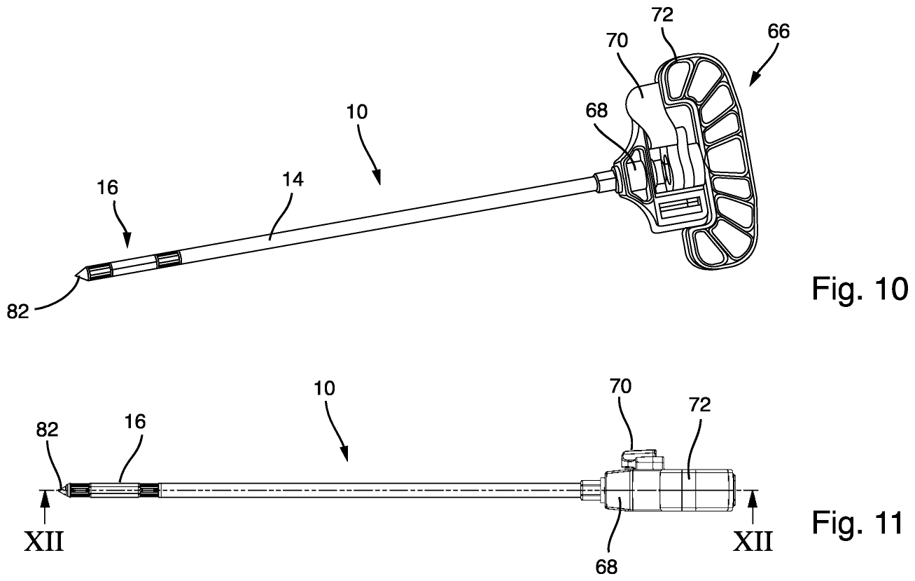

As is clear from FIGS. 1 and 1b, the support body 18 has two ring-like or sleeve-like end portions 22 and 24 which are moved toward one another along the longitudinal axis 27 when the support body 18 is transferred into the expansion position. The end portion 22 is the distal end portion, and the end portion 24 is the proximal end portion on which an insertion cannula 14 (shown in FIG. 10) of an insertion device 10 (shown there) can be arranged.

The support body 18 shown has a total of six carrier portions 26 running in the axial direction between the end portions 22 and 24. The carrier portions 26 are designed to be web-like or band-like and resilient, so that they bulge radially outward when shifted into the expansion position. Instead of six carrier portions 26, three, four, five, seven or more carrier portions 26 may also be provided.

As is clear from FIG. 1 and FIG. 1c, the carrier portions 26 each have counter-latching portions 28 on their inner side facing a longitudinal axis 27. According to FIG. 1a, the counter-latching portions 28 are designed as several directly adjacent latching webs running transversely to the longitudinal axis 27, which webs are introduced into the carrier portions 26. However, the counter-latching portions 28 may also be designed as rectangular recesses, impressions, corrugations, saw teeth, pawls, or the like.

As is clear from FIGS. 1 and 1b, the support body 18 also comprises radially inner, resiliently deformable support portions 30 which move radially outward when the pressure balloons 20 expand and are pushed with their radially outer sides against the radially inner sides of the carrier portions 26. As is clear from FIGS. 1a and 1b, the support portions 30 have tooth-like latching portions 32 on their radially outer side facing the carrier portions 26, which latching portions latch in the expansion position shown in FIG. 1 on the counter-latching portions 28 of the carrier portions 26 and thus fix the support portions 26 in the expansion position in a latching manner, as a result of which the support body 18 is stabilized overall.

When the pressure balloons 20 are dilated, i.e., when the support body 18 is shifted into the expansion position, the latching portions 32 slide along the counter-latching portions 28, with the latching being such that secure latching is ensured in every expansion position. If the pressure balloon 20 is further dilated and the support body 18 expands further as a result, a further relative movement takes place between the carrier portions 26 and the support portions 30, which relative movement is indicated by the arrows 24 in FIG. 1a. As a result, secure latching can be ensured at every stage of the expansion of the support body 18. The carrier portions 26 are consequently securely fixed in their corresponding expansion position by the support portions 30 latching onto the carrier portions 26.

Consequently, a type of free-running locking mechanism is implemented, with the result that the carrier portions 26 are allowed to move in the free-running direction, i.e., into the expansion position, but a return movement of the carrier portions into the insertion position is blocked due to the latching.

The height restoring device 16 shown in FIG. 1 and the support body 18 thereof are designed in two layers, the inner part 50 being shown in FIG. 1b and the outer part 52 being shown in FIG. 1c.

As is clear from FIG. 1b, the inner, one-piece inner part 50 is formed by two inner end portions 22.1 and 24.1, the support portions 30, and an intermediate portion 36 designed in the manner of a ring or sleeve, which intermediate portion, like the end portions 22.1 and 24.1, is not expanded when transferred into the expansion position. The intermediate portion 36 consequently divides the individual support portions 30 into two partial support portions 30.1 and 30.2, each of which has a latching portion 32 in the central region. To shift the inner part 50 into the expansion position, the pressure balloons 20 are each placed between an end portion 22.1 and 24.1 and the intermediate portion 36 and are widened. The provision of the intermediate portion 36 contributes to a relative movement taking place between the carrier portions 26 and the support portions 30 during expansion, the latching portions 32 shifting outward in the radial direction accordingly, and the end portions 22.1 and 24.1 moving toward one another.

As is clear from FIG. 1c, the outer part 52, which is also designed in one piece, is formed by two outer end portions 22.2 and 24.2, the carrier portions 26, and by a structure 53 in the form of connecting webs 54 located between the end portions 22.2 and 24.2 and the carrier portions 26.

In the insertion position shown in FIG. 2, the diameter of the insertion head 12 or the height restoring device 16 is substantially the same as, or only slightly larger or smaller than, the diameter of an insertion cannula 14 shown in FIG. 10 for inserting the height restoring device 16 which is located at the proximal end or can be arranged at the end portion 24. As a result, the entire insertion apparatus 10 having the height restoring device 16 can be safely inserted into one or between two vertebral bodies.

As is clear from FIG. 1, 1c, and 2, two connecting webs 54.1 and 54.2 (54.1′ or 54.2′; 54.1″ or 54.2″) which, in the insertion position, extend in the axial direction are provided between each carrier portion 26 and the corresponding end portion 24. As shown in FIG. 1c, the connecting webs 54.2 and 54.1′ and 54.2′ and 54.1″ etc. of respectively adjacent carrier portions 26 are connected to one another via connection points 55.

It is also conceivable that the partial portions of the connecting webs 54.3 lying between the respective end portions 24.2 and 24.1 and the connection points 55 and running parallel in FIG. 1c are designed in one piece and thus form common connecting webs.

It is also conceivable for the structure 53 to be designed differently, for example in the manner of expanded or support mesh.

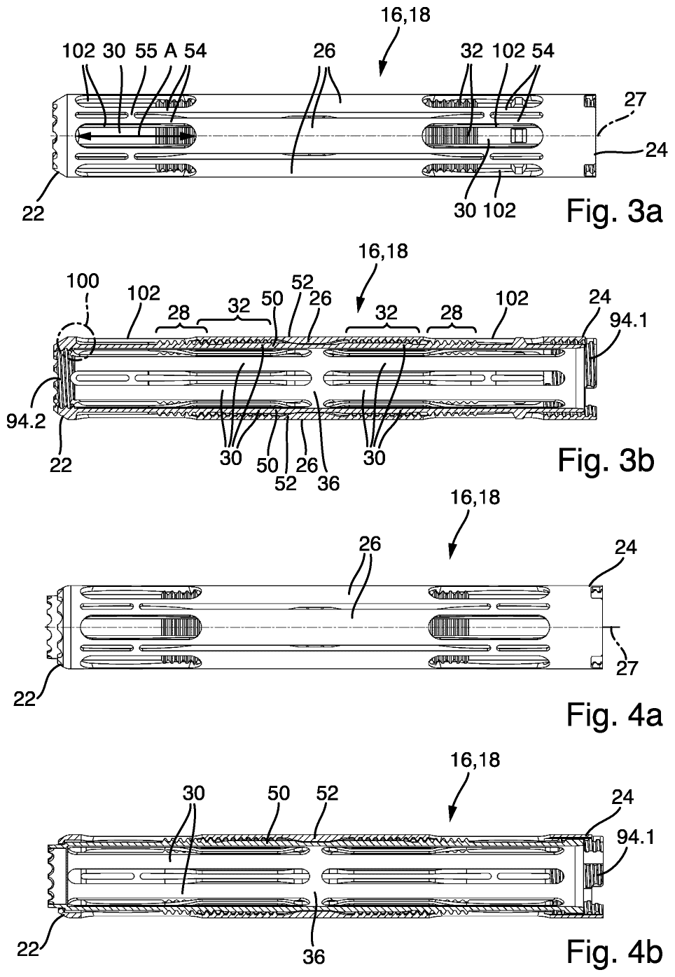

As is clear from FIG. 1 and FIG. 1c, the provision of the connection points 55 causes the connecting webs 54 to spread apart when shifted into the expansion position, as a result of which the distance A between the carrier portions 26 and the end portions 22, 24 is shortened overall and thus a relative movement between the carrier portions 26 and the support portions 30 is supported. The relative movement ultimately results in the progressive latching of the respective latching portions 28 with the respective counter-latching portions 32 during the expansion. The distance A has the greatest value in the insertion position, as shown for example in FIG. 2 or FIG. 3A. The distance A has the minimum value in the expansion position, as shown for example in FIG. 1 or FIG. 1c.

As is particularly clear from FIG. 1b, the provision of the ring-like intermediate portion 36 ensures that forces F acting perpendicularly to the central axis 27 are deflected into the force components F₁ which can ultimately be dissipated via the support body 18. The force deflection is advantageous because the angle α between a plane E1 running perpendicular to the longitudinal axis 27 and the partial support portions 30.1, 30.2 adjoining the intermediate portion 36 is an acute angle which in the expansion position has a value of preferably 30-60° and more preferably about 45°. Correspondingly, the angles β between the planes E2 running perpendicularly to the longitudinal axis 27 and the partial support portions 30.1, 30.2 adjoining the end portions 22, 24 are formed as acute angles, the value of which substantially corresponds to the value of the angle α. Overall, this results in a high degree of stability of the height restoring device 16.

FIG. 1b shows a fixing screw 90 which can be introduced after the expansion position has been reached in order to secure the expansion position. The cavity created by the support body 18 can then be filled with filling material. However, it is also conceivable for the expansion position to be permanently secured with the fixing screw 90, in which case the cavity does not have to be filled with filling material. To introduce the fixing screw 90, the latter may provide an outer thread 92, and the inner part 50 and/or the outer part 52 may have a complementary inner thread 94.1/94.2 on the corresponding end portion 22, 24 thereof, as shown for example in FIG. 3b or FIG. 4b.

In the embodiment of a height restoring device 16 shown in FIGS. 3a and 3b—which substantially corresponds to the embodiment according to FIG. 1 and in which the corresponding components have corresponding reference signs—it is provided that the inner part 50 and the outer part 52 are made in one piece. The end portion 22.1 of the inner part 50 is formed in this case on the end portion 22.2 of the outer part 52 in the region 100. Consequently, no relative movement between the inner part 50 and the outer part 52 when shifted into the expansion position takes place in the region of the end portion 22. The largest relative movement between the inner part 50 and the outer part 52, when shifted into the expansion position, takes place in the region of the end portion 24. In the section according to FIG. 3b, the inner threads 94.1 and 94.2 into which the fixing screw 90 can be screwed can also be clearly seen.

It is also clear from FIG. 3b that the latching portions 28 in the insertion position are substantially located in recesses 102 which are each delimited by two adjacent connecting webs 54.

FIGS. 4a and 4b show a further embodiment of a height restoring device 16, in which, in contrast to the embodiment according to FIGS. 3a and 3b, the outer part 52 is designed as a separate component in relation to the inner part 50. The outer part 50 is arranged in the region of the end portions 22.2 and 24.2 thereof on the end portions 22.1 and 24.1 of the inner part 50 in a limitedly axially shiftable manner. Internal stresses between the inner part 50 and the outer part 52 can thereby be reduced when shifted into the expansion position. In contrast to the embodiment according to FIGS. 3a and 3b, only an inner thread 94.1 for the fixing screw 90 is provided in the end portion 24 of the outer part 52 in the embodiment according to FIGS. 4a and 4b.

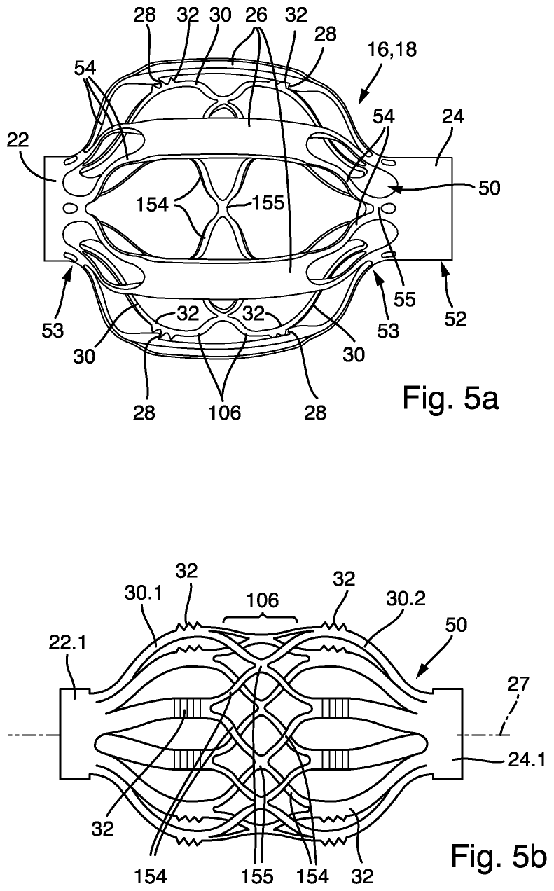

In the embodiments shown in FIGS. 5a and 5b, in contrast to the embodiments according to FIGS. 1, 3a, and 4a, a height restoring device 16 or an associated support body 18 is shown, which support body can be shifted into the expansion position shown in FIG. 5a using only one balloon. The components corresponding to the previous embodiments are marked with corresponding reference signs.

The design of the carrier portions 26 having the connecting webs 54 and the connection points 55 substantially corresponds to the design as shown in FIG. 1 and FIG. 1c. The connecting webs 54 are spread apart at the connection points 55 when shifted into the expansion position.

As can be seen from FIG. 5b, which only shows the inner part 50 of the height restoring device 16, the support portions 30 each have at least two partial support portions 30.1 and 30.2 which each provide latching portions 32. An intermediate portion 106 is provided between the partial support portions 30.1 and 30.1, which intermediate portion is expanded radially outward less than the partial support portions 30.1 and 30.1 when it is transferred into the expansion position. The intermediate portion 106 is formed by connecting webs 154, two connecting webs 154 each which, in the insertion position, extend in the axial direction being provided on each partial support portion 30.1 and 30.2, and the connecting webs 154 of respectively adjacent partial support portions being connected to one another via connection points 155. In the insertion position, the connecting webs 154 lie next to one another in a circular path about a longitudinal axis 27. It is also conceivable that the intermediate portion 106 is designed differently and in particular in the manner of a support or expanded mesh.

The provision of an intermediate portion 106 of this kind allows the connecting webs 154 to be spread apart when shifted into the expansion position, as a result of which the distance between the partial support portions 30.1 and 30.2 is shortened overall and thus a relative movement between the carrier portions 26 and the support portions 30 during the movement into the expansion position is supported. The relative movement ultimately results in the latching of the respective latching portions 32 with the respective counter-latching portions 28.

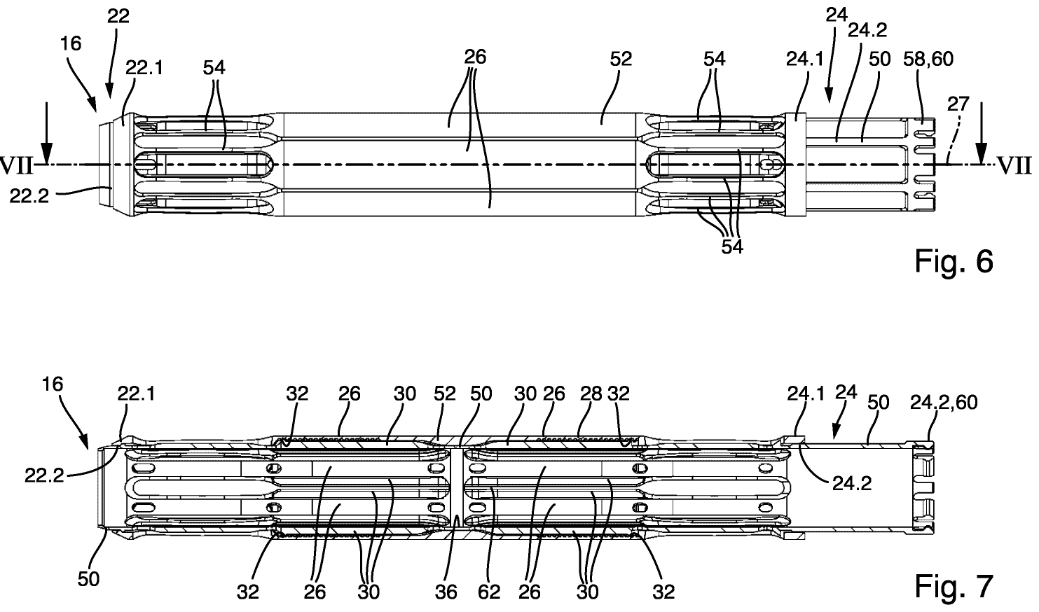

FIGS. 6 to 8 show a further height restoring device 16 for restoring the height of and stabilizing the spinal column, the structure substantially corresponding to the structure of the height restoring device 16 according to FIG. 1, with corresponding components having corresponding reference signs. The height restoring device 16 comprises a sleeve-like inner part 50 and an outer part 52 surrounding the inner part 50 and also being designed like a sleeve.

FIGS. 6 and 7 show an insertion position of the height restoring device 16, in which the height restoring device 16 can be arranged in a compression-fractured vertebral body or between adjacent vertebral bodies and, once there, can be transferred from the insertion position into an expansion position by means of an expansion apparatus.

A support body 18 with a distal and a proximal end portion 22, 24 is provided, the end portions 22, 24 being movable toward one another when shifted into the expansion position. The support body 18 or the one-piece outer part 52 thereof has radially outer support portions 26 and two connecting webs 54 each between the end portions 22.1, 24.1 thereof and the carrier portions 26. The carrier portions 26 are designed such that they move radially outward and act against the vertebral bodies when shifted into the expansion position. In contrast to the embodiment according to FIGS. 1 and 5a, no structure narrowing the distance between the end portions 22, 24 and the carrier portions 26 is provided according to FIG. 6-8 in the case of the height restoring device 16.

The support body 18 or the one-piece inner part 50 thereof has—as is particularly clear from FIG. 7—radially inner support portions 30 between the end portions 22.2, 24.2 thereof, each—according to the embodiment shown in FIG. 1—having two partial support portions 30.1, 30.2 having latching portions 32, a ring-like intermediate portion 36 being provided between the respective partial support portions 30.1, 30.2. Correspondingly, the carrier portions 26 have counter-latching portions 28 on their radially inner sides facing the support portions 30, so that, when shifted into the expansion position, the latching portions 32 move relative to the counter-latching portions 28 and act against the counter-latching portions 28 in such a way that they fix the carrier portions 26 in a latching manner.

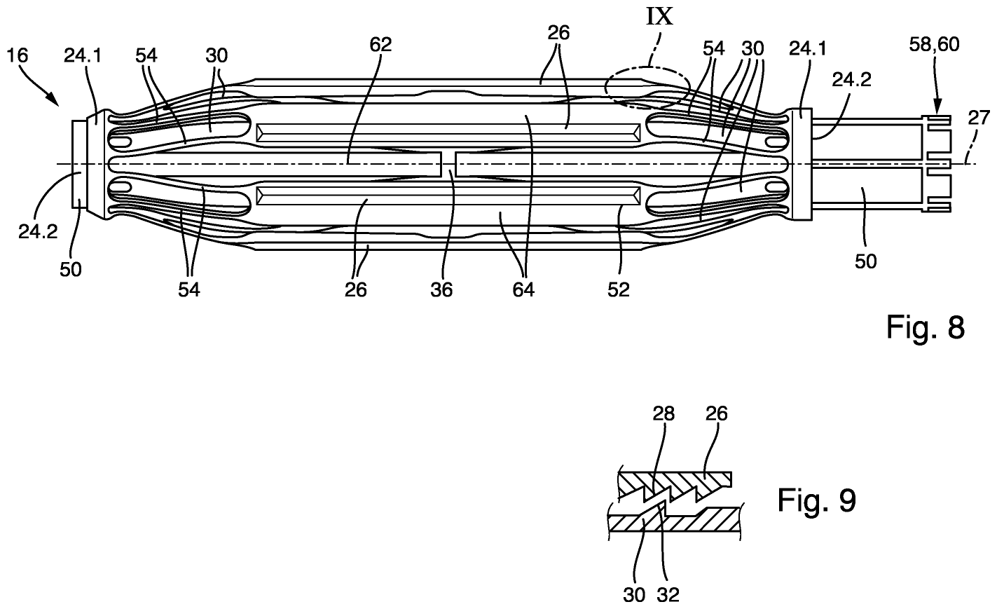

As is clear from FIG. 8, which shows the expansion position of the support body 18, the connecting webs 54 are connected at one end to the distal and proximal ring-like end portions 22.1 and 24.1 of the outer part 52 and at the other end to the carrier portions 24. The connecting webs 54 form desired bending points which deform elastically when shifted into the expansion position. In the axial direction in the region between the connecting webs 54, the carrier portions 26 are designed to be comparatively stable—in contrast to the connecting webs 54—with a comparatively large outer side 64 which overall results in comparatively high stability.

The outer part 52 is also arranged with the end portions 22.1 and 24.1 thereof in an at least conditionally axially shiftable manner on the inner part 50. The carrier portions 26 move radially outward, in particular when shifted into the expansion position, as a result of which the distance between the end portions 22.1 and 24.1 and thus the entire length of the outer part 52 can be shortened. The inner part 50 and the outer part 52 are each provided as one-piece parts that are designed separately from one another.

The inner part 50 has at the proximal end 58 thereof a coupling portion 60 with which the height restoring device 16 can be detachably arranged at the distal end of an insertion cannula 14, as shown in FIG. 10.

As is also clear from FIGS. 6 to 8, the inner part 50 encloses a cavity 62 extending in the axial direction, in which cavity the pressure balloons 20, as indicated in FIG. 1, can be inserted and dilated to shift the height restoring device 16 into the expansion position.

In FIG. 9, which shows the enlarged section IX from FIG. 8 in a sectional view, on the radially outer sides of the support portions 30 facing the carrier portions 26, there can be seen latching portions 32 which interact with counter-latching portions 28 which are provided on the radially inner sides of the support portions 26 facing the support portions 30. The latching portions 32 are designed in this case as singular saw teeth which each interact with a counter-latching portion 28 having several saw teeth in the manner of a free-running locking mechanism.

As is clear from FIGS. 1 to 9, the individual carrier portions 26 and individual support portions 30 are each designed symmetrically to the longitudinal axis 27.

FIGS. 10 to 12 show an insertion apparatus 10 which has a height restoring device 16 at the distal end and an actuating apparatus 66 at the proximal end. The method for restoring the height of and stabilizing the spinal column is therefore as follows:

First, the insertion apparatus 10, as shown in FIG. 10, or the height restoring device 16 thereof, as shown for example in FIG. 1, 2, 3a, 4a, 5a, or 6, is brought into the insertion position thereof, in particular via a catheter placed at the intended expansion site, into a vertebral body or between the vertebral bodies, the height of which is to be restored. However, it is also conceivable for the insertion apparatus 10 to be introduced directly into or between the vertebral bodies, i.e., without the provision of an insertion catheter. After the height restoring device 16 has assumed the precise position thereof, the pressure balloon(s) 20 placed one behind the other in the axial direction within the support body 18 is/are acted upon by a fluid, so that the support body 18 or the carrier portions 26 are transferred from the insertion position into the expansion position. If a plurality of pressure balloons 20 are provided, these can preferably be acted upon independently of one another, so that an optimal alignment of the vertebral body or vertebral bodies is ensured. After the vertebral body or bodies have assumed their optimum position, the pressure balloon(s) 20 is/are removed via or by means of the insertion cannula 14. The support body 18 also retains the expansion position thereof in this case during and after the removal of the pressure balloons 20 due to the latching fixation of the carrier portions 26. In a next step, hardening bone filling material can be introduced into the cavity created by the support body 18, in particular via the insertion cannula 14, for permanent stabilization. The insertion cannula 14 can then be detached from the height restoring device 16 and removed from the body, with the height restoring device 16 then remaining in the body as an implant. Instead of or in addition to the bone filling material, a fixing screw 90, as shown in FIG. 1b, can be introduced to stabilize the support body 18.

In FIGS. 10 to 13, the insertion apparatus 10 assumes an insertion configuration in which it can be inserted into a vertebral body or between two vertebral bodies.

The actuating apparatus 66 comprises three actuating portions 68, 70, and 72. The actuating portion 68 is fixedly arranged on the insertion cannula 14 so that the insertion cannula 14 can be rotated via the actuating portion 68 and moved in the axial direction.

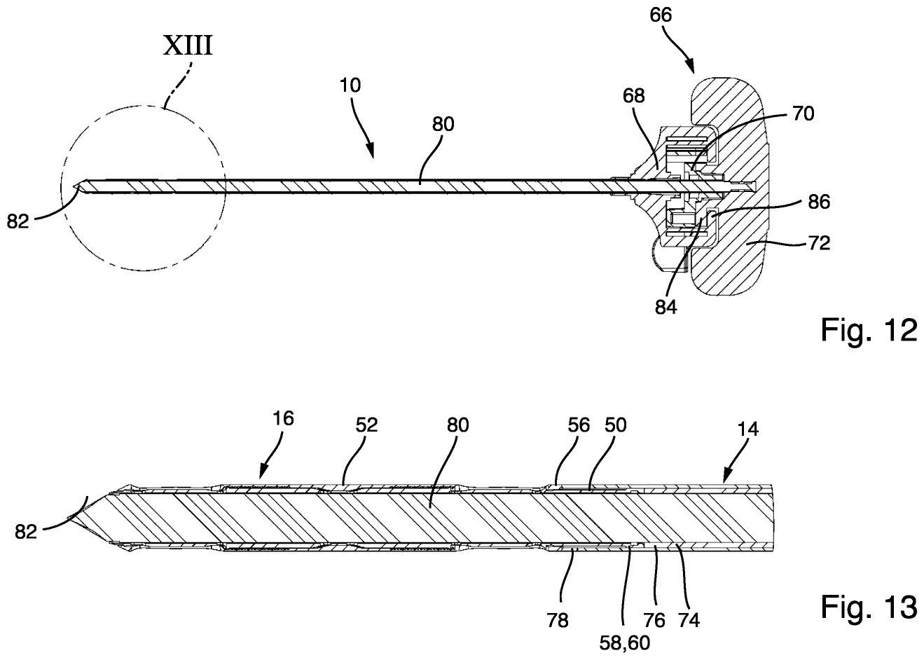

As is clear from the sections according to FIGS. 12 and 13, a shiftable inner cannula 74 is provided on the inner wall of the insertion cannula 14. The proximal end of the inner cannula 74 is movably coupled to the actuating portion 70. The distal end 76 of the inner cannula 74 adjoins the proximal end 58 of the inner part 50 or adjoins the coupling portion 60. As is also clear from FIG. 13, the cannula 14 has a distal end 78 which surrounds the proximal end 58 of the inner part 50 of the height restoring device 16.4, or the coupling portion 60 thereof. As is also clear from FIG. 13, the diameter of the inner cannula 74 corresponds to the diameter of the coupling portion 60, and the diameter of the distal end 78 of the insertion cannula 14 corresponds to the diameter of the proximal end portion 24.2 of the outer part 52.

The insertion apparatus 10 shown in FIGS. 10 to 13 comprises a stylet 80 which can be clearly seen in section in FIGS. 12 and 13 and the proximal end of which is fixedly connected to the actuating portion 72. The stylet 80 extends through the inner cannula 74 and through the cavity 62 formed by the height restoring device 16. The distal end of the stylet 80 provides a cutting tip 82, with which stylet the insertion apparatus 10, or the distal end thereof, can extend into the body to the expansion site, where ultimately the height restoring device 16 is to be used. Such a design has the advantage that no separate insertion catheter—which can also have advantages—is required for inserting the height restoring device 16.

Consequently, the stylet 80, the inner cannula 74, and the insertion cannula 14 can each be actuated independently of one another via the actuating apparatus 66 or the three actuating portions 68, 70, 72 thereof.

To introduce the height restoring device 16 into a vertebral body or between two vertebral bodies, the procedure may be as follows:

First, the insertion apparatus 10, as shown in FIG. 10, is inserted with the distal end thereof, i.e., with the cutting tip 82 first—if such a tip is provided—into the human body until the height restoring device 16 reaches the intended expansion position in a vertebral body or between two vertebral bodies.

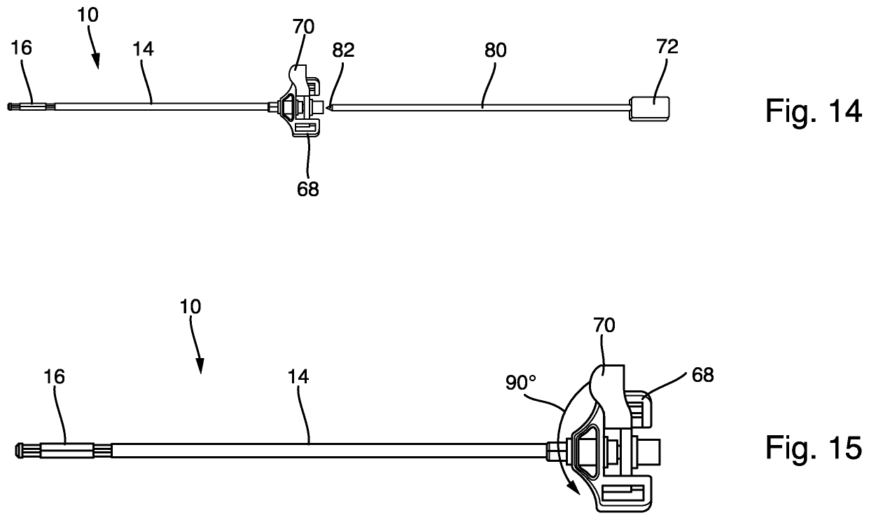

Thereafter, the actuating portion 72 can be rotated in particular by approximately 90° in relation to the actuating portions 68 and 70 in order to detach the axial movement coupling between the stylet 80 and the insertion cannula 14. For this purpose, in the position shown in FIG. 10 and FIG. 12, the actuating portion 72 has an engagement portion 84 which engages behind a holding portion 86 provided on the actuating portion 68. By rotating the actuating portion 72, the engagement portion 84 moves out of the region of the holding portion 86, so that, as shown in FIG. 14, it can be removed together with the stylet 80 in the proximal direction.

In a next step, corresponding instruments can be fed through the inner cannula 74 to the distal end of the insertion cannula 14 or the height restoring device 16. These instruments may be, for example, biopsy devices for taking tissue samples, or endoscopes or video endoscopes.

In order to move the height restoring device 16 into the expansion position, in a next step, pressure balloons 20 are brought through the inner cannula 74 into the cavity 62 of the height restoring device 16. The insertion cannula 14 may comprise or form a line via which the pressure balloons 20 can be brought into the height restoring device 16 and filled with a pressurized fluid, so that the height restoring device 16 or the support body 18 can assume the expansion position. Through the insertion cannula 14, the pressure balloons 20 can also be removed and hardening filling material can be introduced into the expanded support body 18.

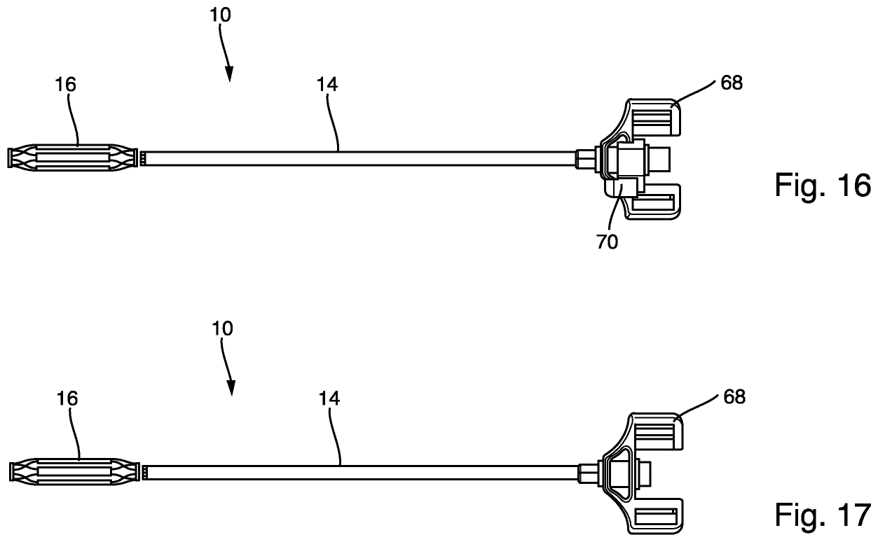

Before the pressure balloons 20 are acted upon with the fluid, as shown in FIG. 16, the actuating portion 70 is rotated by an angle of, in particular, approximately 90° in relation to the actuating portion 68, as a result of which the inner cannula 74 is moved in the distal direction relative to the insertion cannula 14. The actuating portion 70 is coupled to the actuating portion 68 and is positively guided such that, when the actuating portion 70 is rotated, the actuating portion 70 is moved in the distal direction in relation to the actuating portion 68, as a result of which the inner cannula 74 is ultimately also shifted in the distal direction. Due to this distal shifting, in which the distal end 76 of the inner cannula 74, as shown in FIG. 13, acts against the proximal end 58 of the inner part 50 or against the coupling portion 68, the coupling portion 60 is pushed out from the distal end 78 of the insertion cannula 14 in the distal direction. As a result, the height restoring device 16 can ultimately detach from the insertion cannula 14 or from the inner cannula 74, as can be seen in FIG. 16.

In a next step, the pressure balloons 20 can then be acted upon with the pressure fluid, so that the height restoring device 16 assumes the expansion position thereof, as shown in FIG. 16.

After the height restoring device 16 has assumed the final expansion position thereof, the pressure balloons 20 can be removed through the insertion cannula 14 in the proximal direction. Furthermore, the actuating portion 70 together with the inner cannula 74 can also be removed in the proximal direction.

Ultimately, bone filling material or bone cement can be introduced through the insertion cannula 14 into the cavity 62 formed by the height restoring device 16. After the bone filling material has been introduced, the actuating portion 68 can be removed from the body together with the insertion cannula 14 in the proximal direction. However, it is also conceivable for the expansion position to be permanently secured with a fixing screw 90, as shown in FIG. 1b, in which case the cavity does not have to be filled with filling material.

However, before the height restoring device 16 expands, a cavity can first be created in the vertebral body or between the vertebral bodies. If it turns out that the cavity as such is stable enough, the procedure can be in such a way that the cavity is filled directly with bone filling material or bone cement through the insertion cannula 14 without the height restoring device 16 being introduced into the cavity and expanded there. A procedure of this kind has the advantage that it is possible to align and restore the height of the spinal column without the height restoring device 16 remaining in the body.

In order to create such a cavity, the insertion device 10 can be pulled back slightly in the proximal direction after the stylet 80 has been removed, so that a certain cavity remains in the region where the height restoration is to take place. In a next step, one or more pressure balloons 20 can be introduced through the outer cannula 14 into the cavity located in the distal direction behind the height restoring device 16. Thereafter, the pressure balloons 20 can be dilated, as a result of which the height of the vertebral bodies is restored and a cavity is formed in the vertebral body, or between the vertebral bodies. In a next step, the pressure balloons 20 can be removed from the cavity and pulled back into the height restoring device 16 in the distal direction. If the cavity turns out to be stable enough, there is no need to insert and expand the height restoring device 16 into the cavity; bone filling material or bone cement can be introduced directly. However, should the cavity not prove to be sufficiently stable, the height restoring device 16 can be inserted into the cavity by being pushed distally and expanded there before bone filling material is introduced through the insertion cannula 14 into the vertebra or between the vertebral bodies.

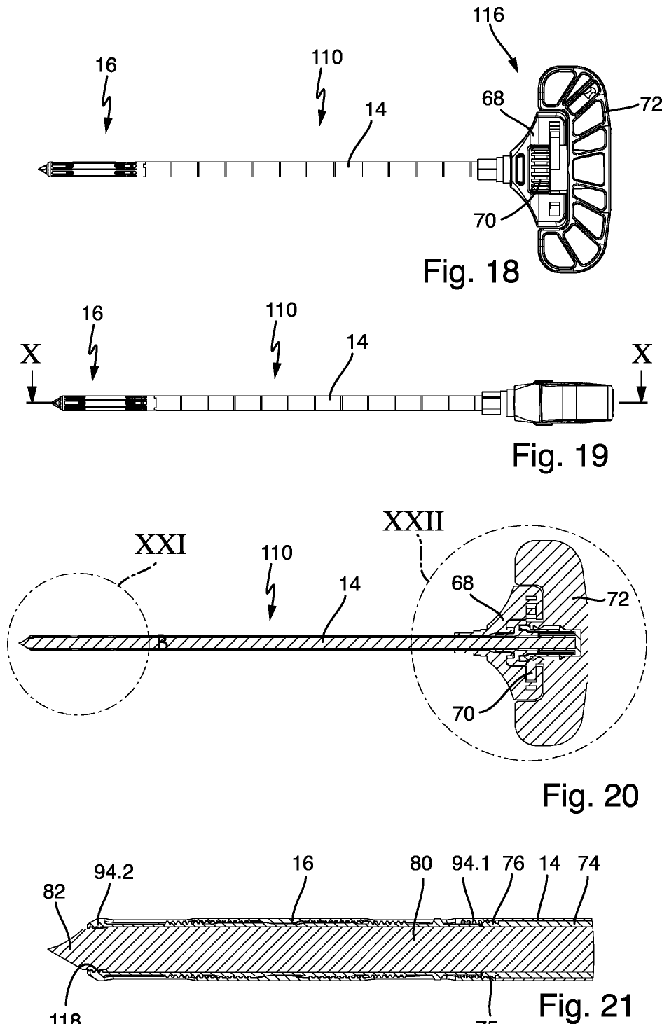

FIGS. 18 to 24 show a further insertion apparatus 110 which has a height restoring device 16 at the distal end and an actuating apparatus 106 at the proximal end. The components corresponding to the insertion apparatus 10 have corresponding reference signs.

The height restoring device 16 shown in FIGS. 18 to 21 is, in particular, a height restoring device 16 shown in FIGS. 3a and 3b. The height restoring device 16 is introduced into the body with the insertion apparatus 110 in the same way as it is introduced with the insertion apparatus 10, as described for FIG. 10-17.

The actuating apparatus 116 comprises, corresponding to the actuating apparatus 66 of the insertion apparatus 10, an actuating portion 68 which is fixedly connected to the outer cannula 14, an actuating portion 70 which is fixedly connected to the inner cannula 74, and an actuating portion 72 which is non-rotatably connected to the stylet 80 via a hexagon 73.

As is clear from FIGS. 18, 23, and 24, the actuating portion 70 is designed as an actuatable rotary wheel, so that the inner cannula 74 is rotated when the actuating portion 70 is actuated. As is clear from FIG. 21, an outer thread 75 is provided at the distal end 76 of the inner cannula 74, which outer thread interacts with the inner thread 94.1 provided at the end portion 24 of the height restoring device 16, as is shown, for example, in FIG. 3b. The height restoring device 16 can consequently be fastened to the inner cannula 74 or to the insertion cannula 14 by rotating the actuating portion 70 in one direction. To detach the height restoring device 16, the rotary wheel of the actuating portion 70 can be rotated in the other direction, so that the outer thread 75 at the distal end 76 is detached from the inner thread 94.1 of the height restoring device 16.

As is also clear from FIG. 21, an outer thread 118 surrounding the cutting tip 82 is provided at the distal end of the stylet 80, which outer thread interacts with the inner thread 94.2 on the distal end portion 22 of the height restoring device 16, as is shown in FIG. 3b. The height restoring device 16 is consequently screwed to the stylet 80 via the thread 118. As a result, when introducing the height restoring device 16, it can be introduced into the body together with the stylet 80 via the actuating portion 72. However, it is also conceivable for the height restoring device 16 to be detached from the inner cannula 74 and thus from the insertion cannula 14 by rotating the actuating portion 70 before the pressure balloons 20 are introduced.

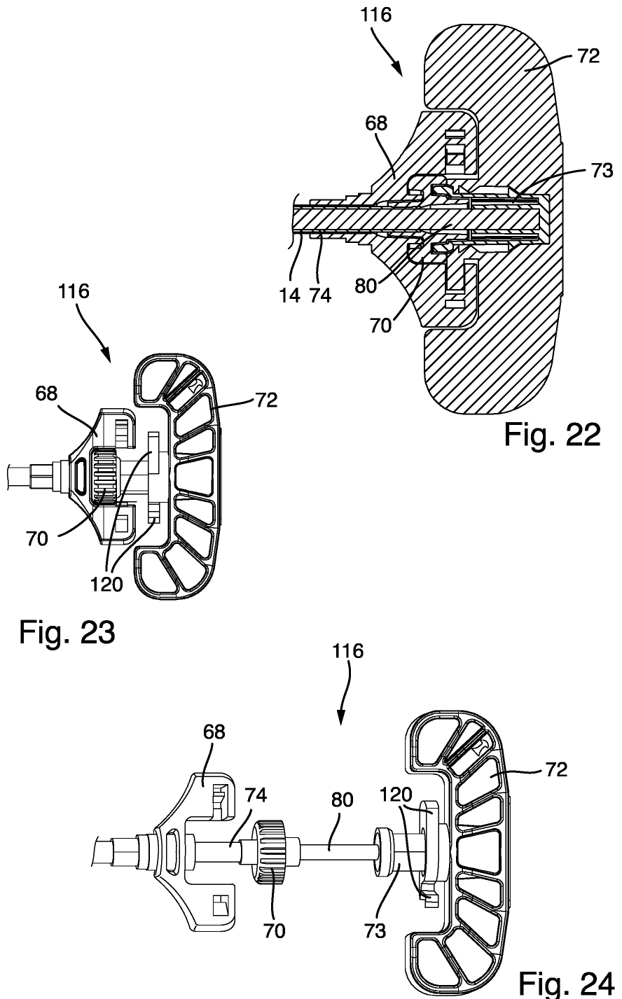

As is clear from FIGS. 22 and 23, the actuating portion 72 acts with the active portions 120 thereof against the actuating portion 68 when the height restoring device 16 is introduced, so that these actuating portions are movably coupled to one another during the introduction process.

After the height restoring device 16 has been placed, as shown in FIG. 23, the actuating portion 72 can be detached from the actuating portion 68 and pulled off axially by rotating in the corresponding direction. Thereafter, the balloons 20 for expanding the height restoring device 16 can be introduced into the height restoring device 16 through the inner cannula 74, as described above for FIGS. 10 to 17. After the expansion of the height restoring device 16, it can be filled with filler and/or permanently secured in the expansion position using a fixing screw 90, as shown in FIG. 1b.

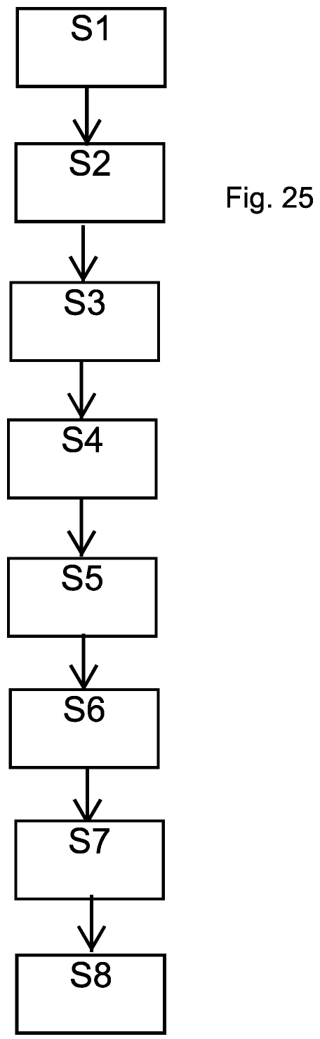

The height restoring device can also be introduced into the expansion site by first creating access to the expansion site according to the following steps S1 to S8, as shown in FIG. 25:

S1: introducing an access instrument, in particular via a pedicle, into the expansion site,

S2: placing a pin guide at the expansion site, in particular through the access instrument, and removing the access instrument,

S3: inserting a drill cannula via the pin guide, the drill cannula comprising a cannula and a drill that can be detached from the cannula, and drilling open the vertebral body at the expansion site,

S4: removing the drill, so that the cannula remains in the body and access to the expansion site is established.

Creating the access first and then inserting the height restoring device through the access is advantageous in particular when the insertion apparatus does not comprise a stylet having a cutting tip, with which stylet the height restoring device can be introduced directly into a vertebral body or between adjacent vertebral bodies.

In this case, the cannula has an inner diameter that is dimensioned so that the height restoring device can be inserted through the access formed by the cannula into the expansion site.

Furthermore, according to S5, it may be provided that at least one pressure balloon is inserted through the cannula into the expansion site and is dilated to widen the expansion site and to create a cavity before the height restoring device is inserted into the access. After the expansion site has been widened and the at least one pressure balloon has been removed, the height restoring device can be inserted according to S6 through the access into the expansion site. After the insertion of the height restoring device into the expansion site, pressure balloons placed in the height restoring device can be dilated according to S7 for transferring the height restoring device into the expansion position, and the pressure balloons can be removed according to S8 after the height restoring device has reached the expansion position. Thereafter, according to S8, bone cement can be introduced through the cannula into the expansion site, and the cannula can be removed.

1. A height restoring device for restoring the height of and stabilizing the spinal column, which device can be arranged in an insertion position in a compression-fractured vertebral body or between adjacent vertebral bodies and once there, can be transferred from the insertion position into an expansion position by means of an expansion apparatus,
comprising a support body which has an inner part arranged around a longitudinal axis, an outer part surrounding the inner part, a distal end portion, and a proximal end portion,
the outer part having carrier portions between the end portions, which carrier portions are designed such that they move radially outward and act against the vertebral bodies when shifted into the expansion position,
the inner part having support portions between the end portions, which support portions have latching portions on their radially outer sides facing the carrier portions, and
the carrier portions having counter-latching portions on their radially inner sides facing the support portions, so that, when shifted into the expansion position, the latching portions move relative to the counter-latching portions and act against the counter-latching portions in the expansion position in such a way that they fix the carrier portions in a latching manner, characterized in that
when the support body is shifted into the expansion position, the end portions move toward one another along the longitudinal axis, and the support portions move radially outward relative to the carrier portions, so that their latching portions act against the counter-latching portions of the carrier portions

2. The height restoring device according to claim 1, characterized in that the carrier portions and/or the support portions are designed, at least in portions, as resiliently yielding webs or in a web-like manner.

3. The height restoring device according to claim 1, characterized in that the latching fixation is designed as a free-running locking mechanism such that the latching portions interact with the counter-latching portions during the movement of the carrier portions into the expansion position in such a way that support is achieved by the latching and a movement of the carrier portions back in the direction of the insertion position is blocked already during the widening process of the support body and before the final expansion position is reached.

4. The height restoring device according to claim 1, characterized in that a structure is provided between the carrier portions and the end portions, which structure shortens the distance between the carrier portions and the end portions when shifted into the expansion position.

5. The height restoring device according to claim 4, characterized in that the structure is formed by connecting webs provided between the individual carrier portions and the corresponding end portion.

6. The height restoring device according to claim 5, characterized in that the connecting webs of respectively adjacent carrier portions are connected to one another, or in that adjacent carrier portions at least partially provide common connecting webs.

7. The height restoring device according to claim 1, characterized in that the individual support portions each have at least two partial support portions having latching portions, and in that an intermediate portion is provided between the respective partial support portions, which intermediate portion is designed in such a way that it is not expanded in the radial direction or is expanded less than the partial support portions when shifted into the expansion position.

8. The height restoring device according to claim 7, characterized in that the intermediate portion is formed by a sleeve or a ring.

9. The height restoring device according to claim 8, characterized in that the intermediate portion is formed by connecting webs, at least two connecting webs each which, in the insertion position, extend in the axial direction being provided on the respective partial support portions, and the connecting webs of respectively adjacent partial support portions being connected to one another, or adjacent partial support portions at least partially providing common connecting webs.

10. The height restoring device according to claim 1, characterized in that the expansion apparatus provides pressure balloons that can be placed and filled between the end portions such that, for transfer into the expansion position, the corresponding pressure balloon dilates and that, when the corresponding pressure balloon dilates, the support portions and the carrier portions are moved in the radial direction, and the support portions act in a latching manner against the carrier portions to fix the carrier portions.

11. The height restoring device according to claim 1, characterized in that the proximal and/or the distal end portion has/have a through hole for introducing a fixing screw after the expansion position has been reached.

12. An insertion apparatus comprising an insertion cannula and a height restoring device provided in or at the free end of the insertion cannula, which device can be arranged in an insertion position in a compression-fractured vertebral body or between adjacent vertebral bodies and once there, can be transferred from the insertion position into an expansion position by means of an expansion apparatus,
comprising a support body which has an inner part arranged around a longitudinal axis, an outer part surrounding the inner part, a distal end portion, and a proximal end portion, the outer part having carrier portions between the end portions, which carrier portions are designed such that they move radially outward and act against the vertebral bodies when shifted into the expansion position,
the inner part having support portions between the end portions, which support portions have latching portions on their radially outer sides facing the carrier portions, and the carrier portions having counter-latching portions on their radially inner sides facing the support portions, so that, when shifted into the expansion position, the latching portions move relative to the counter-latching portions and act against the counter-latching portions in the expansion position in such a way that they fix the carrier portions in a latching manner, characterized in that when the support body is shifted into the expansion position, the end portions move toward one another along the longitudinal axis, and the support portions move radially outward relative to the carrier portions, so that their latching portions act against the counter-latching portions of the carrier portions, the height restoring device, in the insertion position, being insertable into a vertebral body or between two vertebral bodies, and the expansion apparatus for the expansion of the support body being actuated through the insertion cannula.

13. The insertion apparatus according to claim 12, characterized in that a shiftable inner cannula is provided on the inner wall of the insertion cannula, which inner cannula is coupled to the height restoring device in such a way that the height restoring device is detached from the insertion cannula by moving the inner cannula in the distal direction.

14. The insertion apparatus according to claim 13, characterized in that the insertion apparatus comprises a stylet having a cutting tip, which stylet can be passed through the insertion cannula and through the height restoring device and the cutting tip of which forms the distal end of the insertion apparatus in an insertion configuration, so that the height restoring device can be introduced directly into a vertebral body or between adjacent vertebral bodies.

15. The insertion apparatus according to claim 12, characterized in that an actuating apparatus is provided at the proximal end of the insertion apparatus, with which actuating apparatus the inner cannula can be shifted in the distal direction to detach the height restoring device, and/or with which the inner cannula can be inserted into the insertion cannula or removed from it and/or with which the stylet can be inserted into the inner cannula and/or can be removed from it, and/or with which the height restoring device can be brought to the expansion site in the body.

16. A method for restoring the height of and stabilizing the spinal column, comprising the steps of providing an insertion apparatus comprising an insertion cannula and a height restoring device provided in or at the free end of the insertion cannula, which device can be arranged in an insertion position in a compression-fractured vertebral body or between adjacent vertebral bodies and once there, can be transferred from the insertion position into an expansion position by means of an expansion apparatus,
comprising a support body which has an inner part arranged around a longitudinal axis, an outer part surrounding the inner part, a distal end portion, and a proximal end portion, the outer part having carrier portions between the end portions, which carrier portions are designed such that they move radially outward and act against the vertebral bodies when shifted into the expansion position,
the inner part having support portions between the end portions, which support portions have latching portions on their radially outer sides facing the carrier portions, and the carrier portions having counter-latching portions on their radially inner sides facing the support portions, so that, when shifted into the expansion position, the latching portions move relative to the counter-latching portions and act against the counter-latching portions in the expansion position in such a way that they fix the carrier portions in a latching manner, characterized in that when the support body is shifted into the expansion position, the end portions move toward one another along the longitudinal axis, and the support portions move radially outward relative to the carrier portions, so that their latching portions act against the counter-latching portions of the carrier portions, the height restoring device, in the insertion position, being insertable into a vertebral body or between two vertebral bodies, and the expansion apparatus for the expansion of the support body-being actuated through the insertion cannula, and introducing the height restoring device into a vertebral body or between two vertebral bodies at an expansion site and transferred from the insertion position into the expansion position.

17. The method according to claim 16, characterized in that, before the expansion of the height restoring device, an access to the expansion site in the body is created, through which access the height restoring device extends to the expansion site.

18. The method according to claim 17, wherein the step of providing access as follows:
introducing an access instrument into the expansion site,
placing a pin guide at the expansion site and removing the access instrument,
inserting a drill cannula via the pin guide, the drill cannula comprising a cannula and a drill that can be detached from the cannula, and drilling open the vertebral body at the expansion site,
removing the drill, so that the cannula remains in the body and access through the cannula to the expansion site is established.

19. The method according to claim 18, comprising the further step of inserting at least one pressure balloon through the cannula into the expansion site and dilating it to widen the expansion site and to create a cavity before the height restoring device is inserted into the access.

20. The method according to claim 18, characterized in that, after the expansion site has been widened and the at least one pressure balloon has been removed, comprising the step of inserting the height restoring device through the access into the expansion site.

21. The method according to claim 16, wherein for transferring the height restoring device into the expansion position, pressure balloons placed in the height restoring device are dilated after the insertion of the height restoring device into the expansion site, and in that the pressure balloons are removed after the height restoring device has reached the expansion position.

22. The method according to claim 21, wherein, after the pressure balloons have been removed, hardening filling material and/or a fixing screw (90) is introduced into the support body (18).

23. The method according to claim 16, comprising the step of pulling back the height restoring device from the expansion position in the proximal direction before the pressure balloons are dilated in the height restoring device, and in that the pressure balloons are introduced into the cavity provided in the distal direction behind the height restoring device and dilated to create a cavity, and in that then either the cavity is filled with filling material immediately after the pressure balloons have been removed or the height restoring device is introduced into the cavity and expanded there.

24. The method according to claim 16, comprising the step of detaching the height restoring device from the insertion cannula before the pressure balloons are dilated in the height restoring device.

25. The method according to claim 16, comprising the step of introducing the insertion apparatus into the expansion site, wherein the insertion apparatus comprises a stylet having a cutting tip, which stylet can be passed through the insertion cannula and through the height restoring device and the cutting tip which forms the distal end of the insertion apparatus in an insertion configuration, so that the height restoring device can be introduced directly into a vertebral body or between adjacent vertebral bodies.
